(19) United States
(12) Patent Application Publication
(10) Pub. No.: US 2026/0157878 A1
(43) Pub. Date: Jun. 11, 2026

(54) SINGLE-USE MEDICAL WARMING BLANKET ASSEMBLY

(71) Applicants: Ericka Arline Alford Fairweather, Fresno, TX (US); Sharon Kentuvia Alford, Richmond, TX (US)

(72) Inventors: Ericka Arline Alford Fairweather, Fresno, TX (US); Sharon Kentuvia Alford, Richmond, TX (US)

(21) Appl. No.: 19/411,558

(22) Filed: Dec. 8, 2025

Related U.S. Application Data
(60) Provisional application No. 63/728,710, filed on Dec. 6, 2024.

(51) Int. Cl. A61F 7/00; A61L 31/04

(52) U.S. Cl. CPC A61F 7/0097; A61F 7/0085; A61L 31/042; A61L 31/048; A61F 2007/0055

(57) ABSTRACT

A warming blanket assembly includes a warming blanket forming a hollow air space for receiving warmed air from a forced air warming unit and a flexible tubular hose having first and second ends. The first end is permanently connected to the warming blanket and opens into communication with the hollow air space for delivering the warmed air into the warming blanket. The second end is connectable to an outlet port of the forced air warming unit. The warming blanket assembly may be used by positioning the warming blanket over a patient, extending the second end of the hose from the warming blanket to the outlet port of the warming unit, connecting the second end of the hose to the outlet port for receiving a supply of forced air, and turning on the forced air warming unit so that warm air is delivered through the hose into the warming blanket.

### BACKGROUND

The present disclosure relates to methods and apparatus for keeping a patient warm in a hospital bed, such as before, during and/or after a surgical procedure.

### BACKGROUND OF THE RELATED ART

A warming blanket is a part of a forced-air warming system that is commonly used in hospitals during surgery to maintain a patient's body temperature and prevent hypothermia. During surgery, a patient's body may lose heat due to the cool environment of the operating room and the effects of anesthesia, which can impair the body's ability to regulate temperature. The warming blanket is placed over or around the patient, depending on the surgical site, and gently circulates warm air through the warming blanket to maintain a suitable body temperature. Maintaining the patient's body temperature is crucial for reducing the risk of certain complications, such as blood clotting issues, delayed wound healing, and infections, while also improving overall recovery outcomes. Medical teams carefully monitor the patient's temperature to ensure safety and effectiveness throughout the procedure.

Discarding the warming blanket of a forced-air warming system after a patient's surgery is essential to maintain hygiene, prevent cross-contamination, and ensure patient safety. During surgery, the blanket may come into contact with bodily fluids, skin particles, or airborne pathogens, creating a potential breeding ground for bacteria and viruses if not properly cleaned or replaced. However, there are other protocols for cleaning and disinfecting the warming blanket hose that delivers the flowing warm air to the warming blanket. Failure to address any of these cleaning protocols can lead to healthcare-associated infections (HAIs), which pose serious risks to subsequent patients. Adhering to strict infection control protocols, including disinfecting the warming blanket hose after every use, helps protect patients and staff while maintaining the integrity and reliability of the warming system for future use.

### BRIEF SUMMARY

Some embodiments provide a warming blanket assembly comprising a warming blanket comprising at least two flexible layers capable of forming a hollow air space between the two layers for receiving warmed air from a forced air warming unit, and a flexible tubular hose that is permanently connected to the warming blanket, wherein the flexible tubular hose has a first end that opens into communication with the hollow air space for delivering the warmed air into the warming blanket and a second end that is connectable to an outlet port of the forced air warming unit.

Some embodiments provide a method comprising spreading out the warming blanket assembly over a patient, extending a second end of the flexible hose from the patient to the forced air warming unit, and connecting a second end of the flexible hose to an outlet port of the forced air warming unit for receiving a supply of forced air.

### BRIEF DESCRIPTION OF THE SEVERAL VIEWS OF THE DRAWINGS

FIG. 1A is a diagram of a system including a forced air warming unit, a standalone patient warming blanket, and a wire-reinforced durable hose for connecting the forced air warming unit to the patient warming blanket.

FIG. 1B is a diagram of the system after the durable hose has been connected between the forced air warming unit and the patient warming blanket.

FIG. 2A is a diagram of an integrated warming blanket assembly including a warming blanket and a hose that have been folded into a stored condition according to one embodiment.

FIG. 2B is a diagram of an integrated warming blanket assembly including a warming blanket and a filament-reinforced hose that is collapsed in a stored condition according to another embodiment.

FIG. 3A is a side view of a permanent connection between a first end of a filament-reinforced hose and the warming blanket.

FIG. 3B is a cross-sectional side view of the permanent connection between the first end of the filament-reinforced hose and the warming blanket shown in FIG. 3A.

FIG. 4A is a cross-sectional side view of a permanent connection between a first end of a foldable hose and the warming blanket.

FIG. 4B is a cross-sectional side view of an adhesive connection between a first collar at an end of a foldable hose and a second collar around an opening into the warming blanket, where the first and second collars are adhesively secured.

FIG. 5A is a side view of a second end of the filament-reinforced hose axially aligned with an outlet port of the forced air warming device.

FIG. 5B is a side view of the second end of the filament-reinforced hose being threaded onto the outlet port of the forced air warming device.

FIG. 5C is a side view of the second end of the filament-reinforced hose fully connected to the outlet port of the forced air warming device.

FIGS. 6A-C and 6E are side views illustrating a second end of the hose connected to the outlet port of the forced air warming device according to various embodiments.

FIG. 6D is a cross-sectional side view illustrating a second end of the hose connected to the outlet port of the forced air warming device according to another embodiment.

FIG. 6F is an end view illustrating a wire pinch clamp securing the second end of the hose to the outlet port of the forced air warming device according to a further embodiment.

### DETAILED DESCRIPTION

Some embodiments provide a warming blanket for use in surgery or other medical or emergency services that is designed to provide effective thermal support while ensuring patient safety and comfort. The warming blanket may be constructed from soft, lightweight, and non-irritating materials, such as medical-grade polyethylene or woven fabrics, which are both durable and resistant to tearing. The blanket's surface is often perforated with small, evenly distributed holes to allow the controlled flow of warm air from the forced-air system. The warming blanket may include adhesive strips or a non-slip backing to ensure secure placement during surgery. Internally, the blanket may incorporate layers that enhance air distribution and maintain uniform warmth. Still further, the warming blanket may be designed to be disposable to reduce infection risk. The specific configuration of the warming blanket may vary depending on the surgical site, with full-body, upper-body, or lower-body designs tailored for specific procedures, ensuring optimal coverage and efficiency. Additionally, a warming blanket may be used in a variety of non-surgical situations or environments, including an emergency room (ER), an intensive care unit (ICU), preparation for an operation (pre-op), recovery from an operation (post-op), and patient rooms. The warming blankets may be useful for both adult and pediatric patients.

A forced air warming unit for use with a surgical warming blanket is a sophisticated device designed to maintain patient body temperature during surgery. The primary components of the forced air warming unit include a heater, a fan, and a thermostat, where these component work together to deliver consistent and controlled warm air. The heater warms the air to a precise temperature ensuring patient safety by preventing overheating or underheating. The fan then circulates the warmed air through a flexible hose to the surgical warming blanket, where the warmed air may be evenly distributed across the patient's body through perforations. The thermostat plays a crucial role in monitoring and adjusting the air temperature based on preset levels or real-time feedback, ensuring the system operates within safe and effective ranges. The forced air warming unit may feature an intuitive control panel or touchscreen that allows medical staff to set the desired temperature, monitor performance, and quickly make adjustments as needed. Furthermore, the forced air warming unit may include a controller that provide one or more alarms to alert users to potential issues, such as airflow blockages or temperature deviations, ensuring optimal patient care during surgery.

Connecting a flexible tube or hose to the end of the rigid tube that forms the forced air outlet port for receiving a supply of forced air can be accomplished using various methods and/or devices. Each method or device for connecting the hose to the outlet port preferably ensures a secure and efficient connection that is air-tight or at least leaves no significant gaps for uncontrolled escape of the warmed air. One connection method is a friction fit, where the flexible tube is slid over the rigid tube's end, relying on a snug fit to prevent air leakage. A friction fit may especially be suitable if the hose has sufficient elasticity to be stretched around the rigid tube, such as a rubber hose with a thin wall. If the hose does not have sufficient elasticity, a wide rubber band may be secured around the end of the hose to hold the second (free) end of the hose tightly against the outer circumferential surface of the rigid tube. For simplicity, an elastic band with hook-and-loop fastener elements on the ends may be pulled tight around the hose and rigid tube before fastening. Alternatively, hose clamps made from metal or plastic may be used in a similar position and tightened with a threaded fastener to press the hose firmly against the circumferential outer surface of the rigid tube that forms the forced air outlet port. Additionally, some embodiments may use a ring lock including helical threads on the outer surface of the rigid tube and mating helical threads on the inner surface of a rigid ring. A threaded connection may be accomplished by extending a thin hose through the rigid ring and then around the outer helical threads of the rigid tube. In this position, the rigid ring is manually threadedly coupled to the rigid tube with the thin hose secured there between. It should be recognized that any of the materials of the hose and the fasteners coming near the forced air outlet port must be able to withstand the elevated temperatures that may be encountered, such as the highest operating temperature of the forced air warming unit and/or a temperature at which the forced air warming unit will shut itself down.

The warming blanket and/or the hose may be made with paper, plastic, aluminum foil, a fabric or woven mesh or netting, rubber, nylon, polypropylene, polyethylene or polyester, without limitation. In one option, the hose is made from a polyester film or sheet, such as Mylar, that is made from polyethylene terephthalate (PET). For example, the polyester film or sheet may be made by extruding molten PET onto a chilled roll drum to form a film, stretching the film in the machine direction and then in the transverse direction, and then heat setting the film to stabilize it. A polyester film or sheet, such as Mylar, is known for its high tensile strength, chemical and dimensional stability, transparency, reflectivity, gas and aroma barrier properties and electrical insulation.

In some embodiments, the hose (without a spiral wire) is capable of being folded flat to take up less space for the purpose of packaging, shipping, and storing. For example, the elongate hose may be folded in various manners, such as in a Z-fold pattern (i.e., zig-zag, fan fold or accordion fold). Immediately before use, the warming blanket and hose may be removed from the packaging that keeps them clean, the warming blanket may be positioned about the patient and the hose may be unfolded so that the free second end may be extended and connected to the outlet port of the forced air warming unit. Turning on the forced air warming unit forces the warm air through the hose and into the warming blanket such that the hose and warming blanket inflate with a slight air pressure. When the patient's surgical procedure is completed, the forced air warming unit is turned off, the warming blanket is removed from the patient and the hose is disconnected from the forced-air port. The entire warming blanket assembly should then be carefully discarded. Because the hose is connected to the warming blanket that is being discarded, the hose of the warming blanket assembly is prevented from being reused. In embodiments having a hose with a spiral filament reinforcement, the hose may be compressed or collapsed along its central axis until the filament is closely packed together (i.e., like a helical spring toy that is not in use).

Some embodiments provide a transitional configuration of the hose to be used in conjunction with existing warming blanket configurations. For example, a user or facility that has an existing inventory of warming blankets for use with an original equipment manufacturer's reusable hose may remove and discard the reusable hose. Then, the transitional configuration of the hose may be connected between the outlet port of the forced air warming unit and the warming blanket. The term “transitional” is used to indicate that this may be a temporary product design or first phase design that enables a user or facility to avoid wasting their current inventory of warming blankets that do not have an integral hose. However, when the existing inventory of warming blankets have all been used, a user or facility would preferably no longer need the transitional configuration of the hose. The transitional hose may be packaged separate from the warming blanket and can have a paper collar that has an adhesive strip around the paper edge to adhere to the blanket. The hose may be made of the same material as the blanket and may be folded flat. The foldability of the hose is beneficial for packaging but is also easier to discard than a hose that is rigid or has a spiral wire reinforcement. For example, the hose material may be as flexible and foldable as the warming blanket. In one option, a hose and a warming blanket may be separate items provided in the same or separate packages, such that the hose and warming blanket may be irreversibly coupled prior to use. In this option, a flat collar at the end of the hose may be connected to a flat collar on an opening to the warming blanket using an adhesive strip with a removable release liner. The adhesive strip may be formed on either or both of the flat collars and the release liner is pulled off before placing the two flat collars in face-to-face contact with the adhesive there between. The nature of this adhesive connection should be permanent in the sense that attempts to separate the two collars would result in an unusable hose and/or warming blanket. For example, the adhesive may not work more than once and/or the adhered collars may lose integrity when separated.

In some embodiments, a warming blanket assembly integrates the hose with the warming blanket. The warming blanket assembly may be considered to a preferred configuration that would be used after the transitional configuration of the hose is no longer needed. The integrated warming blanket assembly does not require the user or facility to manually connect the hose to the warming blanket and does not allow the user or facility to use the warming blanket without the integrated hose. As a result, the integrated warming blanket assembly requires the user to replace the entire warming blanket assembly such that any contamination that may occur on the hose during a patient procedure is eliminated when the warming blanket assembly is discarded.

Embodiments of the hose are not reusable due to being permanently attached to the warming blanket. Furthermore, the hose materials may be sufficiently durable for multiple uses. A transitional configuration of the hose becomes permanently attached to the warming blanket at the point of use just prior to use, whereas an integrated warming blanket assembly is manufactured with the hose permanently attached to the warming blanket. In either case, a second (free) end of the hose must be connected to the outlet port of the forced air warming unit before the warming blanket may be used with a patient and must be removed from the same outlet port after use. A new hose and warming blanket are needed for each patient procedure and the entire warming blanket assembly must be discarded after completion of the patient procedure.

In some embodiments, the end of the hose that attaches to the blanket may be adhered to the paper part of the blanket using an adhesive. For the transitional configuration of the hose, a cardboard collar on an end of the hose may have a strip of adhesive that encircles the opening in the cardboard collar and the adhesive may be covered by a protective plastic film. In preparation for use of the warming blanket, the protective plastic film may be removed to expose the strip of adhesive and then the cardboard collar of the hose may be pressed against the cardboard collar of the warming blanket with only the strip of adhesive there between. Optionally, even the integrated warming blanket assembly may similarly attach the hose to the warming blanket during manufacturing, but the adhesive may be directly applied to the cardboard collars before pressing the collars together without need for a protective film.

The connection between the hose and the blanket may take various forms. For example, the hose, such as a MYLAR (polyester sheet) hose, may be directly connected to the warming blanket with an adhesive. MYLAR is a trademark of Mylar Specialty Films, the manufacturer of MYLAR products. MYLAR is made from a specific type of polyester referred to as polyethylene terephthalate (PET). Specifically, MYLAR is often referred to as “biaxially-oriented polyethylene terephthalate” (BoPET). One suitable adhesive for adhering to Mylar is an acrylic formula. The end of the hose (a flexible material that forms a tubular shape when filled with air) may be configured to have a face-to-face surface for adhering to the warming blanket. In one option, a circular cardboard collar may be adhered to the warming blanket around a forced air inlet opening into the warming blanket. The duct or hose may then be secured to the cardboard collar.

A cardboard collar may include a flat flange area that is adhered to the blanket around a forced air inlet or opening into the interior of the blanket. The cardboard collar or flange may include a tubular portion extending away from the cardboard collar. The hose may be received around the outer circumference of the cardboard tube and may be adhered to the cardboard tube. If the hose includes a spiral wire reinforcement, the cardboard collar may have two concentric cardboard tubes extending therefrom and forming an annular region there between. The end of the spiral wire reinforced hose may be inserted into the annular region and secured with adhesives or mechanical means.

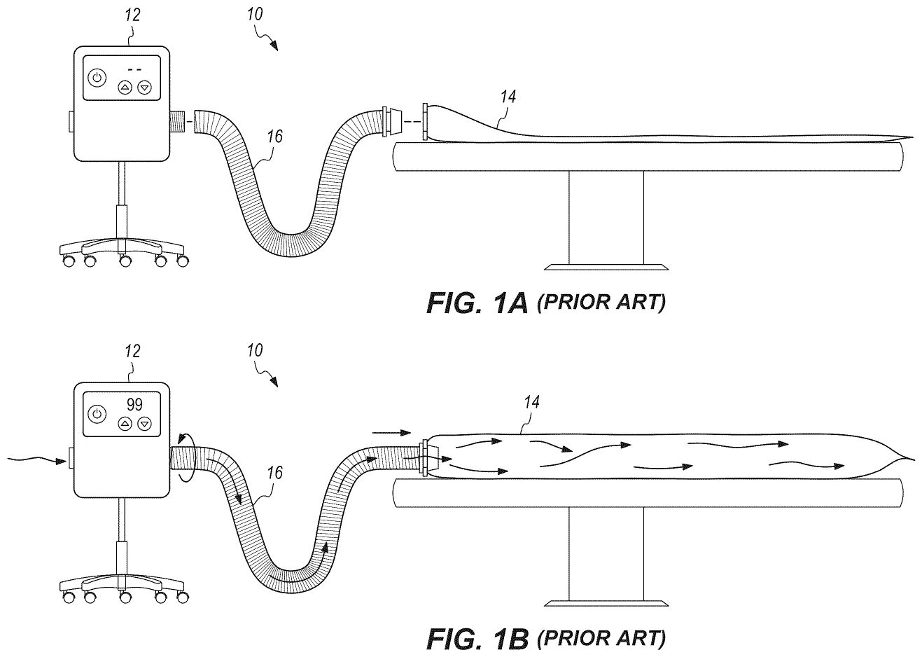

FIG. 1A is a diagram of a system 10 including a forced air warming unit 12, a standalone air warming blanket 14, and a wire-reinforced durable hose 16 for connecting the forced air warming unit 12 to the patient warming blanket 14.

FIG. 1B is a diagram of the system 10 after the hose 16 has been connected between the forced air warming unit 12 and the warming blanket 14. With the forced air warming unit 12 turned on, air is warmed and blown into and through the hose 16 and delivered into the interior of the warming blanket 14.

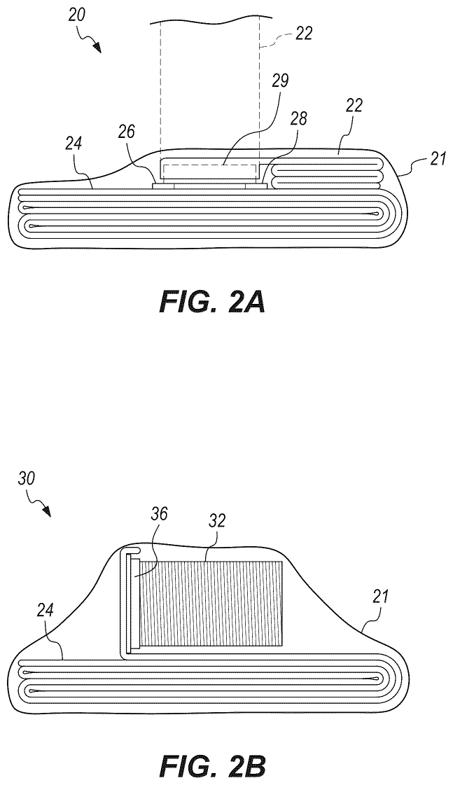

FIG. 2A is a diagram of an integrated warming blanket assembly 20 including a hose 22 and a warming blanket 24 that are both folded into a stored condition. The rigid collar 26 includes a flange portion 28 that is adhered to the warming blanket 24 encircling a forced air inlet opening (not shown) and a tubular portion 29 extending inside the tubular hose 22 (dashed lines used to illustrate an extended position of the hose 22) and adhered thereto. With the warming blanket 24 and the hose 22 in the folded condition, the integrated warming blanket assembly 20 is easily stored in a package 21.

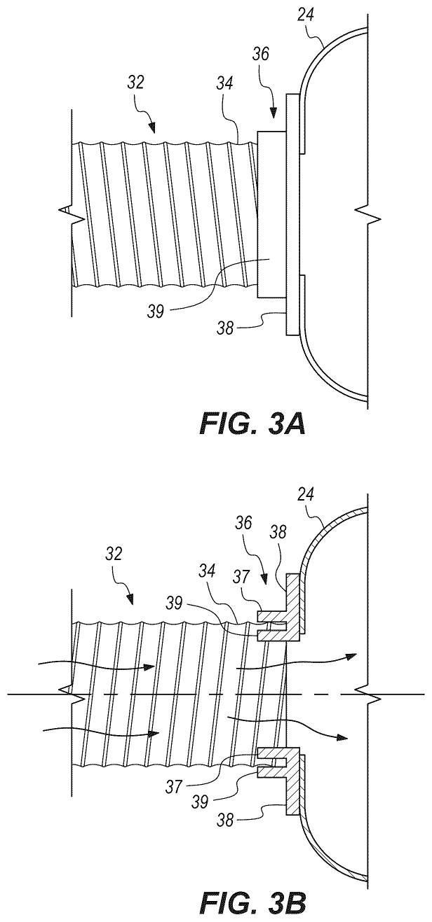

FIG. 2B is a diagram of an integrated warming blanket assembly 30 including a filament-reinforced hose 32 that has been collapsed and laid over on its side in a stored condition. The filament-reinforced hose 32 may be secured to the warming blanket as shown in FIGS. 3A-B.

FIGS. 3A and 3B include a side view and a cross-sectional side view of a permanent connection between a first end 34 of a filament-reinforced hose 32 and the warming blanket 24. The collar 36 includes a flange portion 38 and concentric tubular portions 37 and 39. The first end 34 of a filament-reinforced hose 32 may be received in an annular region between the concentric tubular portions 37 and 39 and secured with an adhesive or other means.

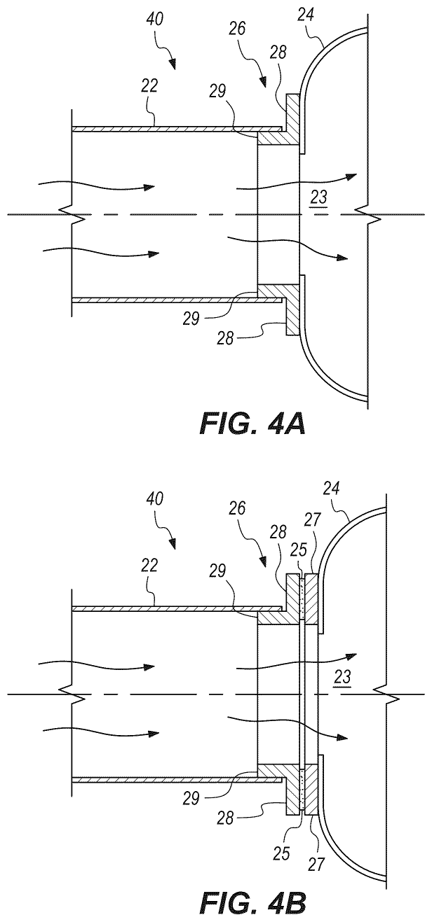

FIG. 4A is a cross-sectional side view of an integrated warming blanket assembly 40 having a permanent connection between the first end of the foldable hose 22 and the warming blanket 24 consistent with FIG. 2A. An adhesive (not shown) is applied between the flange portion 28 of the collar 26 and the material forming the warming blanket 24 near the air inlet opening 23. The same or different adhesive (not shown) may be applied between the tubular portion 29 of the collar 26 and the flexible hose 22, wherein the flexible hose 22 is preferably position over the outer circumferential surface of the tubular portion 29. These uses of the adhesive forms a permanent connection that does not come apart without causing damage to the hose, the collar and/or the warming blanket.

FIG. 4B is a cross-sectional side view of an adhesive connection between a first collar (flange) 28 at an end of a foldable hose 22 and a second collar 27 around the opening 23 into the warming blanket 24, where the first and second collars 28, 27 are secured with an adhesive layer 25. As shown, the first and second collars 28, 27 may be flat and secured together in a face-to-face configuration.

The hose 22 and the warming blanket 24 may be separate items provided in the same or separate packages, such that the hose 22 and the warming blanket 24 may be irreversibly coupled prior to use. In one option, the adhesive may be provided as an adhesive strip with a removable release liner on either or both of the flat collars. The release liner is pulled off before placing the two flat collars 28, 27 in face-to-face contact with the adhesive 25 there between. The nature of this adhesive connection should be permanent in the sense that attempts to separate the two collars 28, 27 would result in an unusable hose 22 and/or warming blanket 24. For example, the adhesive 25 may not work more than once and/or the adhered collars 28, 27 may lose integrity when separated.

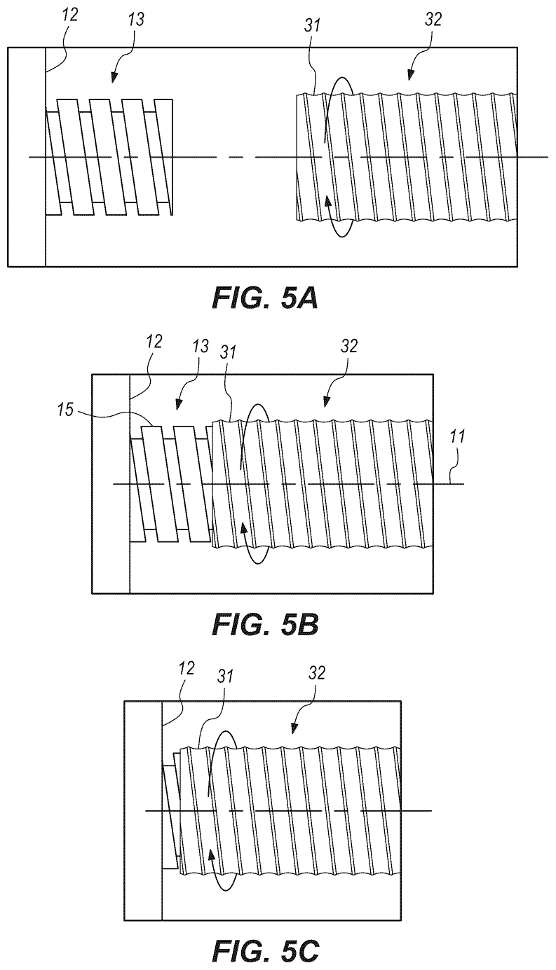

FIG. 5A is a side view of a second (free) end 31 of the filament-reinforced hose 32 axially aligned with a warm air outlet port 13 of the forced air warming unit 12 (see FIGS. 1A-B). FIG. 5B is a side view of the second end 31 of the filament-reinforced hose 32 being turned clockwise about its axis 11 to be threaded onto a set of threads 15 on the outer circumferential surface of the warm air outlet port 13 of the air warming device 12. FIG. 5C is a side view of the second end 31 of the filament-reinforced hose 32 fully and securely connected to the warm air outlet port 13 of the air warming device 12.

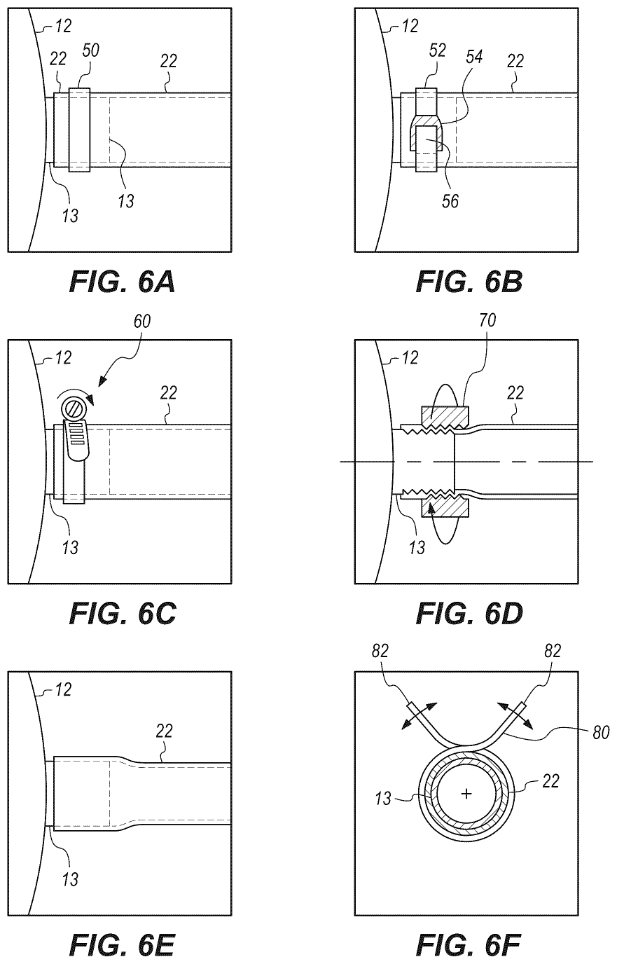

FIGS. 6A-C and 6E are side views illustrating a second (free) end of the flexible hose 22 connected to the outlet port or tube 13. FIG. 6A shows an elastic band 50 that has been stretched and released to apply an elastic force holding the flexible hose 22 tightly against the outer surface of the outlet port 13. FIG. 6B shows a similar elastic band 52, but a first end of the band includes a patch of outwardly facing hooks 54 and a second end of the band includes a patch of inwardly facing loops 56 that are connectable to the hooks 54. FIG. 6C shows a hose clamp 60 tightened about the hose and the outlet port 13. A screw forms a worm gear that engages a series of slots in the band. Turning the screw clockwise causes the band to tighten and turning the screw counterclockwise causes the band to loosen.

FIG. 6D is a side view illustrating a second (free) end of the hose connected to the outlet port using a ring lock system. The outer surface of the outlet port 13 include a set of threads (like a screw) and a rigid ring 70 has an inner surface that includes a mating set of threads (like a threaded hole). Rotating the rigid ring 70 clockwise while the rigid ring is engaging the outlet port 13 causes the threads to engage and draw rigid ring 70 onto the outlet port 13 with the material of the flexible hose 22 securely captured there between. FIG. 6E shows a second (free) end of the hose 22 that has sufficient elasticity to be stretched around the outlet port 13 and then released. Optionally, the outer surface of the outlet port 13 may be textured. FIG. 6F is an end view illustrating a wire pinch clamp 80 securing the second end of the hose 22 to the force air outlet port 13 with the flexible hose 22 disposed there between. Pinching the extended ends 82 of the wire pinch clamp 80 together releases the flexible hose 22 from the outlet tube 13 and releasing the extended ends 82 securely clamps the end of the flexible hose 22 to the outlet tube 13.

The terminology used herein is for the purpose of describing particular embodiments only and is not intended to limit the scope of the claims. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, components and/or groups, but do not preclude the presence or addition of one or more other features, integers, steps, operations, elements, components, and/or groups thereof. The terms “preferably,” “preferred,” “prefer,” “optionally,” “may,” and similar terms are used to indicate that an item, condition or step being referred to is an optional (not required) feature of the embodiment.

The corresponding structures, materials, acts, and equivalents of all means or steps plus function elements in the claims below are intended to include any structure, material, or act for performing the function in combination with other claimed elements as specifically claimed. Embodiments have been presented for purposes of illustration and description, but it is not intended to be exhaustive or limited to the embodiments in the form disclosed. Many modifications and variations will be apparent to those of ordinary skill in the art after reading this disclosure. The disclosed embodiments were chosen and described as non-limiting examples to enable others of ordinary skill in the art to understand these embodiments and other embodiments involving modifications suited to a particular implementation.

What is claimed is:

1. A warming blanket assembly, comprising:
a warming blanket forming a hollow air space for receiving warmed air from a forced air warming unit; and
a flexible tubular hose having first and second ends, wherein the first end is permanently connected to the warming blanket and opens into communication with the hollow air space for delivering the warmed air into the warming blanket, and wherein the second end is connectable to an outlet port of the forced air warming unit.

2. The warming blanket assembly of claim 1, wherein the warming blanket and the flexible tubular hose are constructed with the same material.

3. The warming blanket assembly of claim 2, wherein the material is a medical grade polyethylene.

4. The warming blanket assembly of claim 1, wherein the warming blanket includes at least two flexible material sheets that are secured together to form the hollow air space there between.

5. The warming blanket assembly of claim 1, further comprising:
a collar with a flat flange area that is bonded to the warming blanket around a forced air inlet into the hollow air space and a tubular portion extending from the flat flange area, wherein the first end of the flexible tubular hose is positioned around the tubular portion of the cardboard collar and bonded to the tubular portion of the collar.

6. The warming blanket assembly of claim 1, wherein the warming blanket and/or the hose is made with paper, plastic, aluminum foil, a fabric or woven mesh or netting, rubber, nylon, polypropylene, polyethylene or polyester.

7. The warming blanket assembly of claim 1, wherein the flexible tubular hose includes a flexible wall material and a spiral filament coupled to the flexible wall material to maintain an open tubular passageway through the flexible tubular hose.

8. The warming blanket assembly of claim 7, further comprising:
a collar with a flat flange area that is bonded to the warming blanket around a forced air inlet into the hollow air space and inner and outer concentric tubular portions extending from the flat flange area, wherein the first end of the flexible tubular hose is secured between the inner and outer concentric tubular portions of the collar.

9. The warming blanket assembly of claim 1, further comprising:
a first collar with a first flat flange area that is bonded to the warming blanket around a forced air inlet into the hollow air space; and
a second collar with a second flat flange area and a tubular portion extending from the flat flange area, wherein the first end of the flexible tubular hose is positioned around the tubular portion of the second collar and bonded to the tubular portion of the cardboard collar, and wherein the second flat flange of the second collar is bonded to the first flat flange of the first collar.

10. The warming blanket assembly of claim 9, wherein the first and second collars are made from a cardboard.

11. The warming blanket assembly of claim 1, further comprising:
a fastener for selectively securing the second end of the flexible tubular hose to the outlet port of the forced air warming unit, wherein the fastener is selected from an elastic band, a hose clamp, a threaded ring and a wire pinch clamp.

12. A method of using the warming blanket assembly of claim 1, comprising:
positioning the warming blanket over a patient;
extending the second end of the flexible tubular hose from the warming blanket to the outlet port of the forced air warming unit;
connecting the second end of the flexible hose to the outlet port for receiving a supply of forced air; and
turning on the forced air warming unit so that warm air is delivered through the flexible tubular hose into the warming blanket.

13. The method of claim 12, wherein the second end of the flexible hose is connected to the outlet port by stretching the hose around the outer circumference of the outlet port.

14. The method of claim 12, wherein the second end of the flexible hose is connected to the outlet port by positioning the flexible hose around the outer circumference of the outlet port and then applying a fastener around the hose and the outer circumference of the outlet port, wherein the fastener is selected from an elastic band, a hose clamp, a threaded ring and a wire pinch clamp.

15. The method of claim 12, wherein the flexible tubular hose includes a flexible wall material and a spiral filament coupled to the flexible wall material to maintain an open tubular passageway through the flexible tubular hose, and wherein an outer circumferential surface of the outlet port includes spiral threads, the method further comprising:
connecting the flexible tubular hose to the outlet port by turning the flexible tubular host about its axis so that a portion of the spiral filament at the second end of the flexible tubular hose is securely received by the spiral threads on outer circumferential surface of the outlet port.

16. The method of claim 12, further comprising:
performing a medical procedure on the patient with the warming blanket positioned over the patient and delivering warm air from the forced air warming unit; and then:
turning off the forced air warming unit;
disconnecting the warming blanket assembly from the outlet port or the forced air warming unit; and
discarding the warming blanket assembly before any subsequent use of the forced air warming unit.

17. A method of forming and using the warming blanket assembly of claim 9, comprising:
removing a release liner that is protecting an adhesive layer on the first flat flange and/or the second flat flange to expose the adhesive layer;
pressing the first flat flange face-to-face against the second flat flange with the exposed adhesive layer between the first flat flange and the second flat flange to form the bond the first and second collars together;
positioning the warming blanket over a patient;
extending the second end of the flexible tubular hose from the warming blanket to the outlet port of the forced air warming unit;
connecting the second end of the flexible hose to the outlet port for receiving a supply of forced air; and
turning on the forced air warming unit so that warm air is delivered through the flexible tubular hose into the warming blanket.

18. The method of claim 17, wherein the second end of the flexible hose is connected to the outlet port by positioning the flexible hose around the outer circumference of the outlet port and then applying a fastener around the hose and the outer circumference of the outlet port, wherein the fastener is selected from an elastic band, a hose clamp, a threaded ring and a wire pinch clamp.

19. The method of claim 17, further comprising:
performing a medical procedure on the patient with the warming blanket positioned over the patient and delivering warm air from the forced air warming unit; and then:
turning off the forced air warming unit;
disconnecting the warming blanket assembly from the outlet port or the forced air warming unit; and
discarding the warming blanket assembly before any subsequent use of the forced air warming unit.

20. The method of claim 17, wherein the first and second collars are made from a cardboard that is frangible in response to an attempt to separate the first and second collars are they bonded together with the adhesive layer.
